(19) United States
(12) Patent Application Publication
(10) Pub. No.: US 2022/0079438 A1
(43) Pub. Date: Mar. 17, 2022

(54) PATCH-TYPE THERMOMETER AND SYSTEM THEREFOR

(71) Applicant: AMOSENSE CO.,LTD, Cheonan-si (KR)

(72) Inventors: Hyungil BAEK, Yongin-si (KR); Kyunghyun RYU, Seoul (KR); Beomjin KIM, Siheung-si (KR); Seungyeob YI, Suwon-si (KR); Seongjae AHN, Suwon-si (KR)

(21) Appl. No.: 17/290,743

(22) PCT Filed: Oct. 23, 2019
(86) PCT No.: PCT/KR2019/013951

§ 371 Date: Nov. 29, 2021

(30) Foreign Application Priority Data
Oct. 31, 2018 (KR) 10-2018-0132291

(51) Int. Cl. A61B 5/00; A61B 5/01; G01K 13/20

(52) U.S. Cl. CPC A61B 5/0008; A61B 5/01; A61B 5/002; A61B 5/6833; A61B 2560/0219; G01K 13/20; A61B 2562/0271; A61B 2562/164; A61B 2562/18; A61B 5/742

(57) ABSTRACT

The present invention provides a patch-type thermometer system comprising a body temperature display terminal and a patch-type thermometer. The patch-type thermometer: comprises a flexible printed circuit board, a temperature sensor, an insulating layer attached to the temperature sensor, a heat transfer member for transferring heat to the temperature sensor, a GND region formed at the periphery of the heat transfer member, an exposure hole attached to the temperature sensor, and a protection member; is attached to the skin of a user to measure a body temperature; and transmits a user ID, related app information, and body temperature data in an NFC-based manner. The terminal forms a magnetic field by tagging the patch-type thermometer, requests body temperature measurement, and receives the user ID, the related app information, and the body temperature data from the patch-type thermometer to calculate a body temperature value.

### TECHNICAL FIELD

The present invention relates to a Patch-type thermometer and system therefore.

### DISCUSSION OF THE RELATED ART

In general, for measuring body temperature, we are using a contact type and a non-contact type thermometer, and examples of a general thermometer include mercury, alcohol, and an infrared sensor.

The body temperature can be measured only by waiting for a certain period of time while being worn or in contact with the body using such thermometers.

Accordingly, in the case of infants and patients with discomfort, there is a cumbersome problem in measuring body temperature since it is necessary to help people around them to keep the thermometer worn on the patient's body.

In addition, since the conventional thermometer is a method of checking the body temperature by separating from the user's body after contacting the subject's body for a certain period of time, it is inconvenient to check in real time or periodically.

Accordingly, a patch-type thermometer system has been developed that can be attached to a user's body to conveniently measure body temperature and automatically transmit it to a display terminal to manage body temperature.

However, there has a problem in that body temperature is not measured within an expected time due to structural defects or heat loss of the patch-type thermometer.

### SUMMARY

### Technical Problem

It is an object of the present invention to provide a patch-type thermometer that easily and in a short time measures body temperature by attaching it to the user's body by reducing heat loss and increasing heat transfer rate based on changing the structure of the thermometer.

### Technical Solution

To solve the above problem and defects, the present invention provides a patch-type thermometer system comprising a patch-type thermometer and a display terminal. The patch-type thermometer system is composed of the patch-type thermometer including flexible circuit board on which a Near Field Communication (NFC) antenna pattern for short-range wireless communication is formed on at least one surface, at least one driving chip electrically connected to the antenna pattern for driving the antenna pattern is mounted, a temperature sensor mounted on the upper part of the flexible circuit board to measure body temperature, an insulation layer formed surrounding the temperature sensor, a heat transfer member that is electrically connected to the temperature sensor through a via hole and mounted on the lower surface of the flexible circuit board to directly contact the user's skin, a Ground (GND) region formed around the heat transfer member, and a protection member surrounding the flexible circuit board to prevent exposure of an exposed hole attached to the temperature sensor, the antenna pattern, the driving chip and the temperature sensor; and wherein the patch-type thermometer is attached to the user's skin to measure body temperature, and transmits a user identification (ID), related application program (app) information, and body temperature data through the NFC antenna; a display terminal generating a magnetic field by tagging the patch-type thermometer, requesting body temperature measurement, and receiving the user ID, the related app information, and the body temperature data from the patch-type thermometer to calculate a body temperature value.

In an embodiment of the present invention, wherein in the following equation,

the GND region around the heat transfer member is formed to be expanded to a maximum size in a region excluding the temperature sensor and other peripheral members by increasing A value, which was originally limited by the size of heat transfer member, is increased to the size including the GND region so as to increase an effect of thermal conductivity.

$$\frac{q}{A} ∝ \frac{\partial T}{\partial x} \to \frac{q}{A} = k \frac{\partial T}{\partial x}$$

q=Heat Transfer rate

dT/dx=Temperature gradient in a direction of heat transfer

k=coefficient of thermal conductivity of substance (It is different for each substance and measured through direct experimentation)

In an embodiment of the present invention, the exposed hole attached to the temperature sensor increases a heat transfer rate and is wrapped by the protective member so as not to create a blank space with a skin contact surface.

In an embodiment of the present invention, the patch-type thermometer may be driven by inductive coupling with a magnetic field.

In an embodiment of the present invention, wherein the NFC antenna pattern performs both roles of transmitting data acquired through the temperature sensor and generating driving power required by the driving chip.

$$\frac{q}{A} ∝ \frac{\partial T}{\partial x} \to \frac{q}{A} = k \frac{\partial T}{\partial x}$$

q=Heat Transfer rate

dT/dx=Temperature gradient in a direction of heat transfer

k=coefficient of thermal conductivity of substance (It is different for each substance and measured through direct experimentation)

To solve the above problem and defects, the present invention provides a patch-type thermometer comprising: a flexible circuit board on which a Near Field Communication (NFC) antenna pattern for short-range wireless communication is formed on at least one surface, at least one driving chip electrically connected to the antenna pattern for driving the antenna pattern is mounted; a temperature sensor mounted on the upper part of the flexible circuit board to measure body temperature; an insulation layer formed surrounding the temperature sensor, a heat transfer member that is electrically connected to the temperature sensor through a via hole and mounted on the lower surface of the flexible circuit board to directly contact the user's skin;

a Ground (GND) region formed around the heat transfer member is formed to be expanded to a maximum size in a region excluding the temperature sensor and other peripheral members by increasing A value, which was originally limited by the size of the heat transfer member, is increased to a size including the GND region so as to increase an effect of thermal conductivity, in the following equation, a protection member surrounding the flexible circuit board to prevent exposure of an exposed hole attached to the temperature sensor, the antenna pattern, a driving chip, and the temperature sensor, wherein the patch-type thermometer is attached to the user's skin to measure body temperature, and transmits a user identification (ID), related application program (app) information, and body temperature data through the NFC antenna.

$$\frac{q}{A} ∝ \frac{\partial T}{\partial x} \to \frac{q}{A} = k \frac{\partial T}{\partial x}$$

q=Heat Transfer rate

dT/dx=Temperature gradient in a direction of heat transfer

k=coefficient of thermal conductivity of substance (It is different for each substance and measured through direct experimentation)

In an embodiment of the present invention, wherein the NFC antenna pattern performs both roles of transmitting data acquired through the temperature sensor and generating driving power required by the driving chip.

### Advantageous Effects

According to the present invention, the patch-type thermometer system has the effect of reducing heat loss and increasing the heat transfer rate by changing the structure of the patch-type thermometer. The patch-type thermometer can easily and in a short time measures body temperature by attaching to the user's body.

That is, in the patch-type thermometer, the effect of thermal conductivity can be increased by increasing the region, to which was limited by the size of the heat transfer member, to the GND region according to the applying equation.

### BRIEF DESCRIPTION OF THE DRAWINGS

FIG. 1 is a block diagram schematically showing a patch-type thermometer system including a patch-type thermometer and a display terminal according to embodiments of the present invention.

FIG. 2 is perspective view illustrating the patch-type thermometer in FIG. 1.

FIG. 3 is a bottom view showing a state in which a release film is separated from FIG. 2.

FIG. 4 is a perspective view showing an internal configuration of FIG. 2.

FIG. 5 is a perspective view showing the configuration of a heat transfer member and a GND region in FIG. 3.

FIG. 6 is ‘a-a’ cross-sectional view of FIG. 2.

### DETAILED DESCRIPTION OF THE EMBODIMENTS

With respect to the embodiments of the present invention disclosed in this specification, specific structural or functional descriptions are exemplified for the purpose of describing the embodiments of the present invention only, and the embodiments of the present invention may be implemented in various forms and should not be construed as being limited to the embodiments described in the specification.

Since the present invention can apply various changes and have various forms, specific embodiments are illustrated in the drawings and will be described in detail in this specification.

However, this is not intended to limit the present invention to a specific form of invention, it is to be understood as including all changes, equivalents, or substitutes included in the spirit and scope of the present invention.

Terms such as a first and a second may be used to describe various elements, but the elements should not be limited by the terms. The terms may be used for the purpose of distinguishing one component from another component. For example, without departing from the scope of the present invention, the first element may be referred to as the second element, and similarly, the second element may be referred to as the first element.

When a component is referred to as being “connected” to another component, it may be directly connected the other component, but other components may exist in the middle. On the other hand, when a component is referred to as being “directly connected” to another component, it should be understood that there is no other component in the middle. Other expression describing the relationship between components, such as “between” and “directly between” or “adjacent to” and “directly adjacent to” should be interpreted as well.

The terms used in the application are only used to describe specific embodiments and are not intended to limit the present invention. Singular expressions include plural expressions unless the context clearly indicates otherwise. In the present application, terms such as “comprise” or “having” are intended to designate the presence of a set feature, number, step, action, component, part, or combination thereof, but one or more other features or it is to be understood that the presence or addition of numbers, steps, actions, components, parts, or combinations thereof does not preclude the possibility of preliminary exclusion.

Unless otherwise defined, all terms used herein, including technical or scientific terms, have the same meaning as commonly understood by one of ordinary skill in the technical field to which the present invention belongs. Unless otherwise defined, all terms used therein, including technical or scientific terms, have the same meaning as commonly understood by one of ordinary skill in the technical field to which the present invention belongs. Terms such as those defined in commonly used dictionaries should be interpreted as having meanings consistent with the meanings in the context of the related technology and should be interpreted as ideal or excessively formal meanings unless explicitly defined in this application.

Meanwhile, when a certain embodiment can be implemented differently, a function or operation specified in a specific block may occur differently from the order specified in the flowchart. For example, two consecutive blocks may actually be executed at the same time, or the blocks may be executed backwards depending on a related function or operation.

Hereinafter, exemplary embodiments of the present invention will be described in detail with reference to the accompanying drawings, which will be readily apparent to those skilled in the art to which the present invention pertains. The present invention may be embodied in many different forms and is not limited to the embodiments described herein. In the drawings, parts not relating to the description are omitted for clarifying the present invention, and the same reference numerals are assigned to the same or similar components throughout the specification.

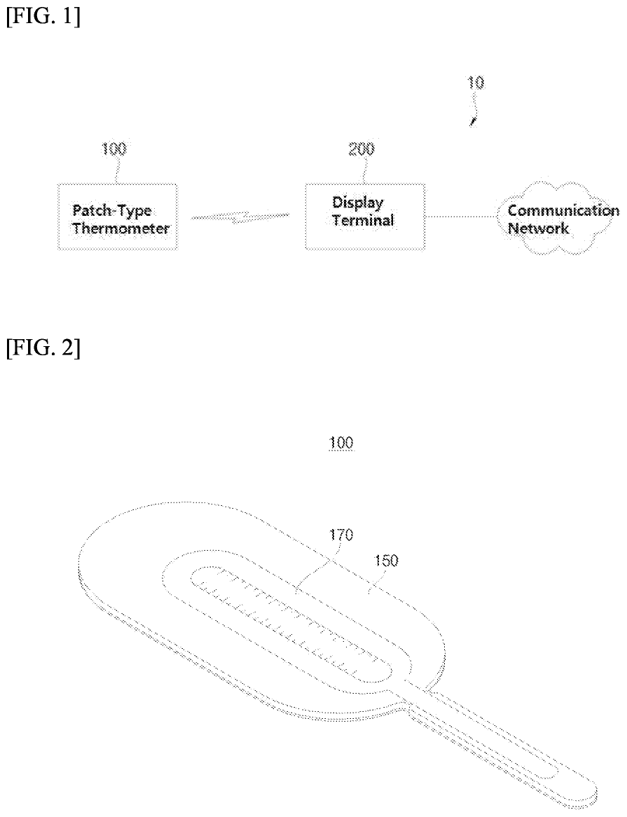

FIG. 1 is a block diagram schematically showing a patch-type thermometer system 10 comprising a patch-type thermometer 100 and a display terminal 200 according to embodiments of the present invention.

The patch-type thermometer 100 may measure body temperature by being attached to the user's skin. The patch-type thermometer 100 may be driven through a Near Field Communication (NFC) antenna. That is, the patch-type thermometer 100 may be driven by inductive coupling with a magnetic field formed from the display terminal 200. The patch-type thermometer 100 may be transmitted a user identification (ID) of the patch-type thermometer 100, related application program (app) information and measured body temperature data to the display terminal 200 through NFC antenna. Here, in the patch-type thermometer 100, a temperature sensor built therein may be an active sensor or a passive sensor.

The display terminal 200 may form the magnetic field by tagging the patch-type thermometer 100, and request the patch-type thermometer 100 to measure body temperature. For example, the display terminal 200 may communication with the patch-type thermometer 100 through NFC antenna. That is, the display terminal 200 may receive the user ID, the related app information and the body temperature data from the patch-type thermometer 100.

In this case, the display terminal 200 may calculates a body temperature value that can be recognized by the user based on the body temperature data received from the patch-type thermometer 100.

Here, the display terminal 200 may a terminal of a user or a guardian, and communicate with the patch-type thermometer 100 through NFC antenna, and may be driven by the related app. The display terminal 200 may perform long-distance communication with a body temperature management server. For example, the display terminal 200 may be a portable electronic device such as a mobile phone, a tablet PC, or a wearable device such as a smart watch, but is not limited thereto.

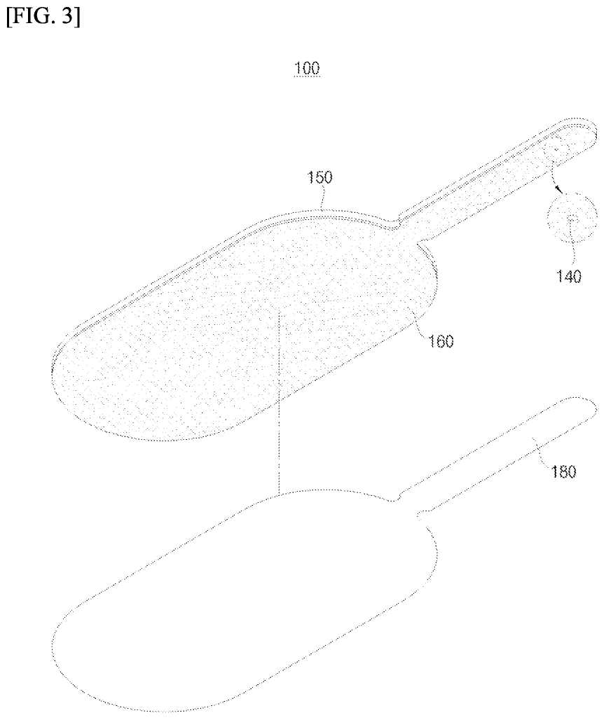

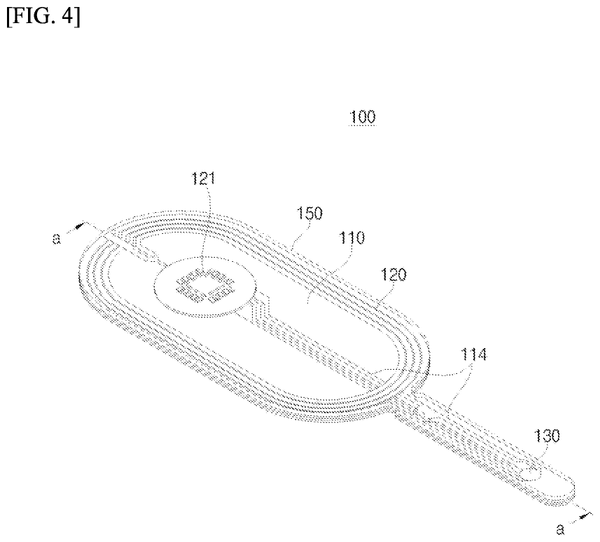

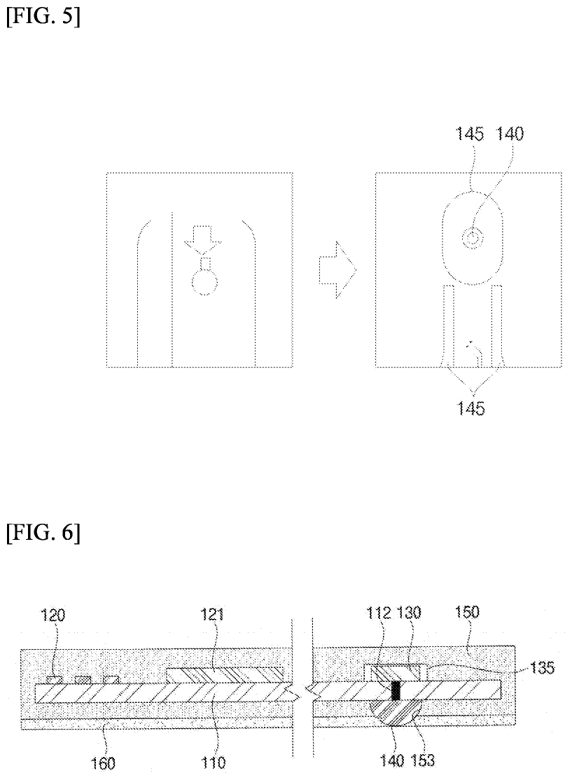

Refer to FIG. 2 to 6, the patch-type thermometer 100 may include a flexible circuit board 110, a temperature sensor 130, an insulation layer 135 attached to the temperature sensor 130, and a heat transfer member 140 for heat transfer to the temperature sensor 130, a GND region 145 formed around the heat transfer member 140, an exposed hole 153 attached to the temperature sensor 130, and a protection member 150.

The flexible circuit board 110 may be a substrate on which various circuit devices are mounted or circuit pattern for electrical connection is formed. For example, the circuit device may be a chipset type device that performs a predetermined function, and the circuit pattern may be an antenna pattern or a wiring pattern for electrical connection.

Such the flexible circuit board 110 may be a known flexible circuit board (FPCB) having flexibility using polyimide (PI) or polyethylene terephthalate (PET).

In this case, the flexible circuit board 110 may have the antenna pattern 120 formed on at least one surface thereof and may be mounted with at least one driving chip 121 electrically connected to the antenna pattern 120. In addition, the temperature sensor 130 may be mounted on one surface of the flexible circuit board 110. The temperature sensor 130 may be electrically connected to with the driving chip 121 through the lead part 114.

For example, the antenna pattern 120 may be an NFC antenna for short-range wireless communication, and the driving chip 121 may be an NFC driving chip that drives the antenna pattern 120.

Accordingly, the antenna pattern 120 may be driven by the driving chip 121 mounted on the flexible circuit board 110 and transmit information acquired from the temperature sensor 130 to the external display terminal 200 through the NFC communication method.

Through this, the body temperature data measured through the temperature sensor 130 may be transmitted to the display terminal 200 through the antenna pattern 120 when NFC tagging with the display terminal 200 is performed.

Meanwhile, the antenna pattern 120 may perform both roles of transmitting data for transmitting information acquired through the temperature sensor 130 and generating driving power required by the driving chip 121.

That is, the antenna pattern 120 may be inductively coupled with a magnetic field formed from the display terminal 200 and may supply power through the inductive coupling to the driving chip 121.

The antenna pattern may perform both roles of transmitting information obtained from the temperature sensor and generating the driving power required by the driving chip 121.

Specifically, the antenna 120 may generate power for driving the driving chip 121 by flexibly combining the magnetic field from the display terminal 200 through the NFC tagging. Here, the generation of power may be referred to as Radio Frequency (RF) harvesting.

Through this, the driving chip 121 may be driven by using the power received from the antenna pattern 120 during NFC tagging. The information obtained through the temperature sensor 130 may be transmitted to the display terminal 200 through the antenna pattern 120.

Accordingly, since the patch-type thermometer 100 does not require a separate power source for driving the driving chip 121, the overall weight may be reduced. The patch-type thermometer 100 may be implemented in an ultra-thin type because the battery may be omitted.

The temperature sensor 130 may generate information related to the user's body temperature by detecting the user's body. Such the temperature sensor 130 may be mounted on one surface of the flexible circuit board 110.

In this case, the temperature sensor 130 may be a digital temperature sensor, and measure the user's body temperature based on heat to be transferred through the heat transfer member 140.

The heat insulation layer 135 may be configured to surround the temperature sensor.

The heat transfer member 140 may be electrically connected to the temperature sensor 130 through the via hole 112 and mounted on the lower surface of the flexible circuit board 110 to directly contact the user's skin.

In the following heat conduction equation,

$$\frac{q}{A} ∝ \frac{\partial T}{\partial x} \to \frac{q}{A} = k \frac{\partial T}{\partial x}$$

q=Heat Transfer rate

dT/dx=Temperature gradient in a direction of heat transfer

k=coefficient of thermal conductivity of substance (it is different for each substance and measured through direct experimentation)

The GND region 145 formed around the heat transfer member 140 may be formed to be expanded to a maximum size in a region excluding the temperature sensor and other peripheral members by increasing A value, which was originally limited by the size of the heat transfer member, to the size of including the GND region so as to increase an effect of thermal conductivity.

Meanwhile, the temperature sensor 130 and the heat transfer member 140 may be electrically connected to each other through the via hole 112.

Accordingly, the temperature sensor 130 may be mounted on the upper surface of the flexible circuit board 110, which is the same surface as the driving chip 121, without having to be exposed to the user's skin side. The temperature sensor 130 may be completely covered by the protective member 150 to improve an airtightness to be described later.

In this case, the driving chip 121 may be disposed inside the antenna pattern 120, and the temperature sensor 130 may be disposed outside the antenna pattern 120, and the driving chip 121 and the temperature sensors 130 may be electrically connected to each other through a lead part 114 formed on at least one surface of the flexible circuit board 110.

In this case, the heat transfer member 140 may be mounted on the lower surface of the flexible circuit board 110 as described above, so that it may directly contact with the user's skin. Accordingly, the body heat transmitted from the user's skin may be transmitted to the temperature sensor 130 through the heat transfer member 140.

To this end, the heat transfer member 140 may be made of a metal material having excellent thermal conductivity.

In this case, the heat transfer member 140 may have a shape capable of maintaining a state in contact with the user's skin. To this end, the heat transfer member 140 may have a shape in which a central portion protrudes convexly in one direction.

For example, the heat transfer member 140 may be formed in a hemispherical shape or a dome shape.

For this reason, when the patch-type thermometer 100 is attached to the user's skin, the central portion of the heat transfer member 140 may always be in contact with the user's skin even if the attachment portion is a curved portion. Accordingly, the heat transfer member 140 may smoothly transfer heat transferred from the user's skin to the temperature sensor 130.

As illustrated in FIG. 6, the heat transfer member 140 may be exposed to the outside through the exposure hole 153 formed in the protective member 150 to be described later. Accordingly, the exposed hole attached to the temperature sensor 130 may increase the heat transfer rate and the area around the exposed hole 153 may be wrapped with the protective member so as not to create a blank space with the skin contact surface.

On the other hand, the patch-type thermometer 100 may include the protective member 150 surrounding the flexible circuit board 110 so as to prevent the antenna pattern 120, the driving chip 121, the temperature sensor 130 from being exposed to the outside.

Accordingly, the protection member 150 may be formed to completely surrounding the rest parts except for the portion corresponding to the heat transfer member 140.

That is, the protective member 150 may be disposed to completely cover the upper and lower surfaces of the flexible circuit board 110, so that the antenna pattern 120, the driving chip 12, the temperature sensor 130 and the flexible circuit board 110 excluding the heat transfer member 140 are to prevent from being exposed to the outside.

In this case, the protective member 150 may be made of a flexible material. Through this, even if the patch-type thermometer 100 is attached to a curved body part, it may be flexibly changed according to the user's body flexion, thereby increasing adhesion with the user's body.

For example, the protective member 150 may have a molding body made of insulating resins such as silicone. However, the protective member 150 may be not limited thereto, and in the form of a sheet formed of a fluoropolymer resin such as polyethylene terephthalate (PET), polypropylene(PP), or polyethylene(PE), or release paper, and any material having airtightness and insulating properties may be used without limitation.

Meanwhile, an adhesive layer 160 may be formed on one surface of the protective member 150 of the patch-type thermometer 100.

The adhesive layer 160 may attach the patch-type temperature meter 100 to the user's body by providing adhesion. Here, the adhesive layer 160 may be formed on the surface where the exposed hole 153 for exposing the heat transfer member 140 to the outside is formed. Accordingly, when the patch-type thermometer 100 is attached to user's skin through the adhesive layer 160, the heat transfer member 140 may directly contact the user's skin.

For example, the adhesive layer 160 may be a gel-type adhesive layer. As the adhesive layer 160 is made of a material whose adhesive strength is restored upon contact with moisture, it may be used repeatedly.

However, it should be noted that the material of the adhesive layer 160 is not limited thereto, and can be used without limitation as long as it can provide adhesive force with the user's skin.

In addition, the patch-type thermometer 100 may have an information display unit 170 formed on one surface of the protection member 150. The information display unit 170 may include at least one or more information of letters, numbers, and figures.

For example, the information display unit 170 may be a logo or a figure for aesthetic sense. Through this, the user can identify information on the product by checking various information through the information display unit 170.

As described above, the present invention has been described with respect to particularly preferred embodiments. However, the present invention is not limited to the above embodiments, and it is possible for one who has an ordinary skill in the art to make various modifications and variations, without departing off the spirit of the present invention.

### EXPLANATION OF THE NUMBER

100: a patch-type thermometer, 200: a display terminal
110: a flexible circuit board, 130: a temperature sensor
135: an insulation layer, 140: a heat transfer member
145: a GND region, 150: a protection member
153: an exposed hole

What is claimed is:

1. A patch-type thermometer system, comprising:
a patch-type thermometer including a flexible circuit board on which a Near Field Communication (NFC) antenna pattern for short-range wireless communication is formed on at least one surface, at least one driving chip electrically connected to the antenna pattern for driving the antenna pattern is mounted,
a temperature sensor mounted on the upper part of the flexible circuit board to measure body temperature,
an insulation layer formed surrounding the temperature sensor,
a heat transfer member that is electrically connected to the temperature sensor through a
via hole and mounted on the lower surface of the flexible circuit board to directly contact the user's skin,
a GND region formed around the heat transfer member, and
a protection member surrounding the flexible circuit board to prevent exposure of an exposed hole attached to the temperature sensor, the antenna pattern, the driving chip and the temperature sensor; and
wherein the patch-type thermometer is attached to the user's skin to measure body temperature, and transmits a user ID, related application program (app) information, and body temperature data through the NFC antenna,
a display terminal generating a magnetic field by tagging the patch-type thermometer, requesting body temperature measurement, and receiving the user ID, the related app information, and the body temperature data from the patch-type thermometer to calculate a body temperature value.

2. The patch-type thermometer system of claim 1, wherein in the following equation, the
GND region around the heat transfer member is formed to be expanded to a maximum size in a region excluding the temperature sensor and other peripheral members by increasing A value, which was limited by the size of heat transfer member, is increased to the size including the GND region to increase an effect of thermal conductivity,
$$\frac{q}{A} ∝ \frac{\partial T}{\partial x} \to \frac{q}{A} = k \frac{\partial T}{\partial x}$$
q=Heat Transfer rate
dT/dx=Temperature gradient in a direction of heat transfer
k=coefficient of thermal conductivity of substance (It is different for each substance and measured through direct experimentation)

3. The patch-type thermometer system of claim 1, wherein the exposed hole attached to the temperature sensor increases a heat transfer rate and is wrapped by the protective member so as not to create a blank space with a skin contact surface.

4. The patch-type thermometer system of claim 1, wherein the NFC antenna pattern performs both roles of transmitting data acquired through the temperature sensor and generating driving power required by the driving chip.

5. The patch-type thermometer system of claim 1, wherein the patch-type thermometer is driven by inductive coupling with the magnetic field of the display terminal.

6. A patch-type thermometer, comprising:
a flexible circuit board on which a Near Field Communication (NFC) antenna pattern for short-range wireless communication is formed on at least one surface, at least one driving chip electrically connected to the antenna pattern for driving the antenna pattern is mounted;
a temperature sensor mounted on the upper part of the flexible circuit board to measure body temperature;
an insulation layer formed in a form surrounding the temperature sensor,
a heat transfer member that is electrically connected to the temperature sensor through a via hole and mounted on the lower surface of the flexible circuit board to directly contact the user's skin;
a GND region formed around the heat transfer member is formed to be expanded to a maximum size in a region excluding the temperature sensor and other peripheral members by increasing A value, which was limited by the size of the heat transfer member, is increased to the size including the GND region to increase an effect of thermal conductivity in the following equation;
$$\frac{q}{A} ∝ \frac{\partial T}{\partial x} \to \frac{q}{A} = k \frac{\partial T}{\partial x}$$
q=Heat Transfer rate
dT/dx=Temperature gradient in a direction of heat transfer
k=coefficient of thermal conductivity of substance (It is different for each substance and measured through direct experimentation); and
a protection member surrounding the flexible circuit board to prevent exposure of an exposed hole attached to the temperature sensor, the antenna pattern, a driving chip and the temperature sensor;
wherein the patch-type thermometer is attached to the user's skin to measure body temperature, and transmit a user ID, related application program (app) information, and body temperature data through the NFC antenna.

7. The patch-type thermometer of claim 6, wherein the NFC antenna pattern performs both roles of transmitting data acquired through the temperature sensor and generating driving power required by the driving chip.
